(19) United States
(12) Patent Application Publication
(10) Pub. No.: US 2010/0240993 A1
(43) Pub. Date: Sep. 23, 2010

(54) METHOD AND MEDICAL APPARATUS FOR DETERMINING A TUMOR CHARACTERISTIC

(76) Inventors: Thorsten Bergsdorf, Strasslach (DE); Christian Chaussy, Strasslach (DE); Ralf Nanke, Neunkirchen am Brand (DE); Stefan Thueroff, Muenchen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/727,357

(22) Filed: Mar. 19, 2010

(30) Foreign Application Priority Data
Mar. 19, 2009 (DE) 10 2009 014 052.2

(51) Int. Cl. A61B 8/00

(52) U.S. Cl. 600/437

(57) ABSTRACT

In a method and medical apparatus to determine a tumor characteristic related to a body region of a patient, the body region is exposed to acoustic energy that mechanically irritates the body region. A first value of a characteristic value of the patient that is correlated with the irritation is determined at a first point in time. A second value of the characteristic value is determined at a second point in time after the first point in time and after the exposure. The tumor characteristic is determined from the change between first value and second value.

### BACKGROUND OF THE INVENTION

1. Field of the Invention

The present invention concerns a method to determine a tumor characteristic and a medical apparatus suitable for implementing such a method.

2. Description of the Prior Art

A diagnosis, which a physician normally makes with regard to a patient, is most often based on a number of characteristics in the form of measurement or characteristic values. These values are determined or obtained at the patient. Examples are medical image data such as x-ray images, MR or CT exposures or also measurement values such as pulse rate, blood pressure, blood composition or characteristic blood values. Characteristics that serve for the diagnosis of a tumor disease at the patient are called characteristic tumor values. Characteristics that deliver characteristic values that are limited to a specific body region of a patient (for example the liver, spleen, prostate, testes or chest are called tumor characteristics “related to this body region”.

These characteristics provide assistance to the physician in his or her diagnosis of whether the appertaining body region is afflicted with a tumor or not. Using the diagnosis, the physician then decides about a suitable therapy to be conducted on the patient. Such tumor characteristics are determined in corresponding methods to be implemented at the patient. These methods therefore are also called diagnosis-supporting or therapy-supporting methods and are designated as diagnostics, together with the diagnosis.

An exact diagnostic is a significant building block in cancer care and therapy of a patient. The earlier and more precisely that a diagnosis can be made, the more efficiently that the therapy introduced based on the diagnosis can be designed.

Moreover, the less expensive, safer and less invasive that such a method is, the more probable its application in medical practice.

In particular for prostate carcinomas (PCa), an imaging diagnostic is less efficient and expensive. The decision of whether a PCa is present in a patient, or even if lymph nodes of the patient have already metastasized, is an important criterion for the subsequent therapy.

The previous gold standard to establish lymph node metastases is a histology. In addition to this operative lymph node diagnostic, for some time it has also been known to use very expensive and complicated imaging methods such as contrast agent-enhanced magnetic resonance tomography (MRT) or positron emission tomography/computer tomography (PET-CT).

Among other things, one important building block in the PCa diagnosis is the determination of the concentration of prostate-specific antigen (PSA concentration) as a tumor characteristic. PSA is a marker that can be measured in serum for prostate tissue that is not specific for PCa. Prostate cancers are connected in approximately 90% of all cases with a increased PSA value.

The tumor characteristic must thus deliver an optimally differentiated statement that the physician uses in his decision of whether a specific body region of a patient is afflicted with a tumor or not. The decision of whether a suspicion of cancer exists or not using a measured PSA value is difficult for the physician since the PSA values of healthy patients and patients afflicted with cancer can often differ only slightly. This also applies for other serum markers that are used as tumor characteristics or in connection with tumor characteristics, for example alpha-fetoprotein (AFP) or beta human chorionic gonadotropin (bHCG). AFP and b-HCG are not tissue-specific and thus cannot deliver reliable conclusions of the primary tumor; a massive b-HCG or β-HCG increase is also found given the presence of a pregnancy, for example.

### SUMMARY OF THE INVENTION

It is an object of the present invention to provide an improved method to determine a tumor characteristic and a medical apparatus suitable for this purpose.

The invention is based on the insight that prostate tissue outputs PSA into the bloodstream given mechanical irritation, for example as occurs in a digital rectal examination. The PSA value measured in the blood serum of the patient temporarily increases due to the pressure on the tissue. Outside of the fascia of prostate, only malignant prostate tissue can be found. Here as well a PSA increase should be demonstrable via a mechanical irritation, as in the intraprostatic prostate cells.

The invention is based on utilization of the fact established above for an improved determination of a tumor characteristic. The basis is to implement a direct and targeted mechanical irritation of the organ to be observed or, respectively, the appertaining body region of the patient. In other words, a specific body region of a patient is mechanically irritated so as to release a serum marker in a higher concentration than otherwise. The concentration of the serum marker in the blood is therefore artificially increased. This is therefore a stimulus diagnostic method. The artificially induced rise in the concentration of the serum marker in the blood of the patient allows the physician to make a more certain pathological finding or, respectively, diagnostic conclusion from the appertaining tumor characteristic.

The invention is based on generating the mechanical irritation by the introduction of acoustic energy into the appertaining body region in order to provoke the cells in the irritated area to dump factors (for example tumor markers) that are detectable in the bloodstream. This should function in all tumors (for example prostate cancer, testicular cancer, breast cancer, etc.) that express tumor markers at a primary organ.

In the method according to the invention, the body region is irradiated with acoustic energy. The acoustic energy serves to mechanically irritate the body region. In other words, the body region is charged with a mechanical stimulus. At a first point in time, a first value of a characteristic value of the patient is then determined. The characteristic value is correlated with the irritation. In other words, from the patient a characteristic value is determined that is amplified, attenuated or emphasized in some other way relative to the unstimulated state by the mechanical irritation of the body region. The body region can be irradiated in a pulsed manner, or continuously.

At a second point in time, a second value of the same characteristic value is then determined. The second point in time is after the first point in time and also after the point in time of the exposure of the body region with acoustic energy (thus after the irritation). The tumor characteristic is then determined from the change of the characteristic value between the first point in time and the second point in time, thus from the change between the first value and the second value. The first value should be defined as a base value before the irritation. Other procedures are also possible, however, as is explained below.

In the method according to the invention a targeted stimulus of the body region (for example of an organ or of lymph nodes) is thus conducted in order to amplify a characteristic value for diagnostic purposes with regard to its significance as a tumor characteristic, for example to increase a serum marker release.

The characteristic value is advantageously a value that can be determined from a bodily fluid of the patient, for example a characteristic blood or urine value.

The method offers a novel form of care or early diagnosis and acquisition of information in the time curve of the characteristic value (for example the serum marker curve) and the level of a measurable amplitude, for example in the time curve of the characteristic blood value. The method offers an elegant, non-invasive, fast and inexpensive determination of the characteristic tumor value, for example for lymph node diagnostics. Operations to obtain histological samples thus are not necessary. The amplified change of the values of the characteristic value that is artificially caused by the mechanical irritation is only temporary; for example, a PSA increase is no longer detectable 24 hours after the mechanical irritation. The method according to the invention thus can be repeated within the scope of further diagnoses, for example in order to implement a course monitoring of the patient. The method can be transferred from the cited PCa/PSA example to many other medical fields. The method according to the invention is also called extracorporeal shock wave diagnostics (ESWD) given the use of shock waves for irritation.

In an embodiment of the method, one of the values is determined approximately one hour after the exposure. In other words, first point in time or second point in time occurs approximately one hour after the exposure of the body region with acoustic energy. According to experience, the characteristic value (for example PSA) rises to a maximum approximately one hour after the irritation of the body region, such that in this case a maximum value for the characteristic value can be determined. The method according to the invention is therefore suitable for ambulant operation.

If the first point in time is approximately one hour after the exposure, the second point in time is thus selected so late that a decrease of the second value relative to the first value is already observed again, for example.

It is suggested to place the first point in time so that a reference value is determined therefrom as a base value. In an advantageous embodiment, the first value is therefore determined before the exposure. The first point in time therefore lies before the exposure, and the first value serves as a reference value for the second value measured after the exposure according to the invention. For example, the maximum increase of the characteristic value relative to a base value can thus be determined.

Numerous possibilities exist for the determination of the tumor characteristic from the values, of which advantageous examples are as follows.

In an embodiment of the method, the tumor characteristic is determined as a quotient of second and first value. The tumor characteristic then indicates by what multiple of its initial value or, respectively, reference value the characteristic value was increased by the irritation.

In another embodiment of the method, in addition to first value and second value additional values (thus multiple measurement values) are determined at different points in time, and the tumor characteristic is determined as a time curve of the values. If the points in time are close enough to one another, the time curve representing an increase or decline of the characteristic value can be graphically presented before, during or after the mechanical irritation. The tumor characteristic is then again the maximum superelevation of the curve above a base value, the slope of the curve at specific points, rise or fall times of the characteristic value to specific percentiles of the maxim, for example.

In a preferred embodiment of the method, a tumor marker in blood serum (for example PSA, AFP or bHCG is determined as a characteristic value as explained above.

As was also explained above, in a further embodiment of the method the tumor characteristic for the prostate, the testicles or the mamma is determined as a body region.

In a further embodiment of the method, the body region is exposed with acoustic energy in the form of diagnostic or focused ultrasound, shock waves or pressure waves. The cited methods are particularly well suited to introduce mechanical energy into specific body regions of the patient that are to be examined. With the method according to the invention, a new application possibility for already existing apparatuses (such as lithotriptors, thus shock wave applicators) results in addition to the previously known use in therapy. This leads to a multiple use of the apparatuses, and therefore to a cost advantage.

In a further variant of the method, microbubbles in the body region or in immediate proximity to the body region are destroyed, whereby the body region is irradiated with acoustic energy. This method variant uses an effect that is otherwise undesirable in ultrasound therapies (that, for example, can lead to tissue damage in the patient) in a controlled manner.

The microbubbles are generated by, for example, a systemically administered ultrasound contrast agent and then destroyed in a controlled manner by irradiation with ultrasound.

In a further embodiment of the method, the body region is exposed with acoustic energy from a source placed percutaneously or transrectally on the patient. Both methods are non-invasive and are particularly suitable in connection with the application of the method to the prostate, for example.

The above object also is achieved by a medical apparatus according to the invention is suitable for the execution of the method according to the invention that is described above. The medical apparatus thus has essentially already been explained in connection with the method according to the invention. The medical apparatus has a source for acoustic energy and an evaluation unit with an input via which the evaluation unit receives values of the characteristic value of the patient that are determined at the first and second point in time. The evaluation unit is fashioned to determine the tumor characteristic from the changes between the first and second values.

In a preferred embodiment, the medical apparatus has an imaging apparatus that serves to localize the body region and to align the source on the body region. The medical apparatus can therefore be operated without support from additional apparatuses and can nevertheless find the intended body region exactly and implement the method according to the invention at this body region.

In a preferred embodiment, the imaging apparatus is an ultrasound imaging apparatus, which is harmless to the patient with regard to radiation exposure.

In a further embodiment, the medical apparatus moreover has an in vitro testing unit to determine the value of the characteristic value of the patient. The characteristic value or can therefore be determined directly in the medical apparatus. Particularly for characteristic values from a bodily fluid (for example urine or blood samples of the patient), the fluid does not need to be first sent to a laboratory for evaluation in order to be able to further use the determined values subsequently in the method according to the invention.

A first field of application for the method according to the invention is the care or initial diagnosis as a screening test. At the first point in time—before the application of energy in the body region—the “tumor marker baseline” is measured. After the irritation—for example of the prostate—the post-irritation value of the tumor marker and the quotient of both values is determined. An above-average increase of the value (thus a high quotient) can indicate to the physician (within the scope of his or her diagnosis) a carcinoma or at least an increased risk of such, for example. Alternatively, the time curve of the tumor marker can also be measured. It is also possible to implement the irritation more often by repeated energy application, possibly with increasing acoustic energy. Alternatively, the decay of the temporary increase of the tumor marker can be determined as a tumor characteristic.

A further field of application is substitutive diagnostics in operative procedures. Instead of an operative lymph node removal to identify a tumor, an application of acoustic energy can ensue, for example to the lymph nodes in the pelvis. In the case of a PSA-expressing tumor, the presence of a metastasis could be precluded or proven by evaluating the tumor characteristic generated with the method according to the invention within the scope of a diagnosis. A significant increase of the PSA value after an irritation of the lymph discharge paths or iliac lymph nodes with acoustic energy thus supports the diagnosis of a physician in an unambiguous and non-invasive verification of lymph node metastases of a prostate cancer.

### BRIEF DESCRIPTION OF THE DRAWINGS

The single FIGURE schematically illustrates a medical apparatus in use on a patient in the execution of the method according to the invention.

### DESCRIPTION OF THE PREFERRED EMBODIMENTS

The FIGURE shows a patient 2 on whom an ESWD is conducted. The medical apparatus 4 used for this comprises a source 6 for acoustic energy 8 (represented by a beam cone) and an evaluation unit 10. In the example a physician would like to generate a tumor diagnosis for the body region 12 (the prostate of the patient 2 in the example) and uses the method according to the invention in order to determine a tumor characteristic on which he can then base his diagnosis.

Before beginning the ESWD, a bodily fluid is extracted from the patient (in the example blood 16 from his bloodstream 14) at a point in time t1, and from this a first value 18a of a characteristic value B is determined (in the example as a characteristic blood value of the value of the concentration of PSA 20 at 0.41 ng/ml.

The body region 12 is subsequently irradiated (struck) with focused acoustic energy 8 by the perineally placed source 6, whereupon this releases more PSA 20 into the blood stream 14. The irradiation is conducted with an energy 8 of 5.3 J in 722 individual shots with a shock wave strength from 0.1 to 1. Of the entire volume of the body region 12 (approximately 30 ml in the case of the prostate), only two foci (thus target points or target areas) each in a respective lobe of the prostate are irradiated.

In order to specifically reach the body region 12 with energy 8, the medical apparatus 4 has an imaging apparatus 22. During the irradiation a localization of the body region 12 is implemented with said imaging apparatus 22. A contrast agent depiction of the prostatic urethra and a radioscopy in the manner of a cystourethogram (CUG) are conducted.

At the point in time t2, a second value 18b of the characteristic value B (thus the concentration of PSA 20 in the blood) is determined again at 0.47 ng/ml. The PSA value was thus increased by 15% by the bombardment. The point in time t2 is placed approximately one hour after the introduction of the energy in order to establish a maximally increased value of PSA.

The values 18a, b are determined in a laboratory in a first embodiment. The values returned by the laboratory are then supplied to an input 11 of the evaluation unit 10. The tumor characteristic K is then determined as a quotient of the values 18b to 18a: K=0.47/0.41=1.146. This value then serves the physician as a basis for his diagnosis of whether a prostate cancer exists.

In an alternative embodiment, the medical system 4 contains an in vitro test unit 13 that determines the values 18a-c from the blood 16.

After the ESWD, a distinct macrohematuria and dysuria are established in the patient. This is an additional indication that the shock waves have safely arrived from the source 6 at the prostate 12.

In an alternative embodiment, the body region 12 is exposed substantially over its total area so that it is “shock” by the energy 8. Accordingly more PSA 20 is thereby released and the value 18b rises further in relation to the value 18a.

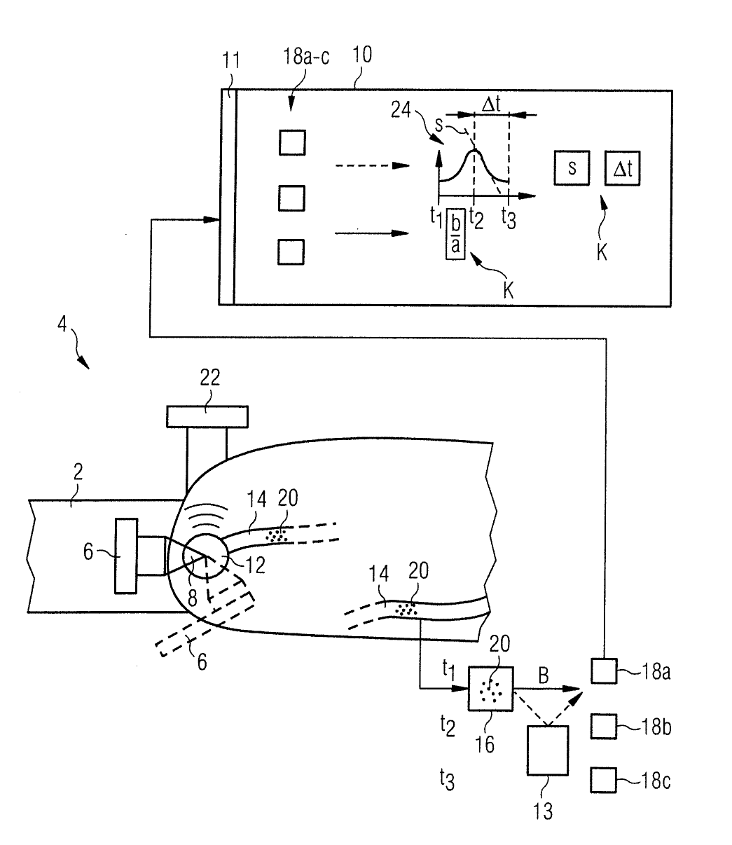

In the perineal ESWL shown in FIG. 1 (thus the placement of the source 6 at the perineum of the patient 2), due to the bone window in the form of the ischial bump it is however difficult to bombard the prostate, especially in the region near the rectum. Bone pain, analysis pain and pelvic floor cramps are possible side effects. Under the circumstances the perineum is therefore not suitable as an access path for the ESWL.

FIG. 1 shows an alternative source 6 which is introduced transrectally into the patient in order to emit the energy 8 on a different path to the body region 12.

In another embodiment, the source 6 is an endorectal transrectal ultrasound (TRUS) scanner that operates at a frequency of 7.5 MHz. The scanner is externally fixed by a mount running in the analysis cleft and is covered with a balloon. Such a fluid-filled balloon with a volume of less than 150 cm² (for example) can be varied in volume.

In an alternative embodiment, this TRUS scanner also serves as an imaging apparatus: the scanner generates slice images in transversal slices image acquisition a cranio-caudal scan process. Given a duration of approximately 15 s, it then calculates an organ model and depicts it on a screen (not shown). The source 6 can thus be used simultaneously for imaging.

It is thus also possible to retrofit a known TRUS scanner for stimulus diagnosis or, respectively, for a double function: for this the scanner must merely be replaced with an applicator which can generate a correspondingly higher sound pressure, for example an ARFI applicator (Acoustic Radiation Force Imaging). Equipped with a conventional TRUS scanner, the source 6 can then simultaneously function as an intraoperative navigation system.

The required stimulus energy in the form of the energy 8 is hereby approximately equal to that given perineal access due to the spatial proximity; however, it is possible that less energy needs to be applied since less absorption and scattering occurs. In the example it has turned out that the energy “injection” into the body region 12 should be fan-shaped, for example with a fan width of 2 cm. The energy injection should be unfocused and the effective penetration depth should be 5 cm at maximum.

At a further point in time t3, 24 hours after the application of the energy 8, an additional value 18c in the blood 16 of the patient is determined which again corresponds to the initial value 18a, meaning that the artificially increased value of PSA 20 is then no longer detectable.

In different embodiments the imaging apparatus thus contains two transducers or a single transducer that can then be operated with different energies for imaging and exposure.

In an alternative embodiment, the values 18a-c acquired at the points in time t1-t3 and a plurality of intermediate values of the characteristic value B are shown as time curve 24 in the form of a diagram over time. The tumor characteristic K is then likewise determined as a change between the individually determined values of the characteristic value B. In this case, for example, as a slope s of the decaying curve of the characteristic blood value after the point in time t2 or as a time interval Δt as a time between the maximum of the curve 24 until the characteristic value B has decreased again to the original value measured at the point in time t1.

Although modifications and changes may be suggested by those skilled in the art, it is the intention of the inventor to embody within the patent warranted hereon all changes and modifications as reasonably and properly come within the scope of their contribution to the art.

We claim as our invention:

1. A method to determine a tumor characteristic related to a body region of a patient, comprising the steps of:
exposing the body region to acoustic energy to mechanically irritate the body region;
determining a first value of a characteristic value of the patient that is correlated with said irritation, at a first point in time;
determining a second value of said characteristic value at a second point in time following said first point in time and after exposing the body region to said acoustic energy; and
in a processor supplied with said first and second values, automatically determining a tumor characteristic from a change between said first value and said second value.

2. A method as claimed in claim 1 comprising determining said first and second values of said characteristic value from a bodily fluid of the patient.

3. A method as claimed in claim 1 comprising selecting said first point in time or said second point in time to be approximately one hour after exposing the body region with said acoustic energy.

4. A method as claimed in claim 1 comprising determining said first value before exposing said body region of the patient with said acoustic energy.

5. A method as claimed in claim 1 comprising, in said processor, determining said tumor characteristic as a quotient of said second value and said first value.

6. A method as claimed in claim 1 comprising determining at least one additional value of said characteristic value at an additional point in time, and determining said tumor characteristic in said processor from a time curve represented by said first value, said second value and said additional value.

7. A method as claimed in claim 1 comprising employing a tumor marker in blood serum of the patient as said characteristic value.

8. A method as claimed in claim 7 comprising selecting said tumor marker from the group consisting of PSA, AFP and bHCG.

9. A method as claimed in claim 1 comprising irradiating a body region of the patient with said acoustic energy, selected from the group consisting of prostate, testicles and breasts.

10. A method as claimed in claim 1 comprising irradiating said body region with acoustic energy in a form selected from the group consisting of diagnostic ultrasound, focused ultrasound, shockwaves, and pressure waves.

11. A method as claimed in claim 1 comprising irradiating the body region with acoustic energy by destroying microbubbles in or proximate to said body region with acoustic energy.

12. A method as claimed in claim 1 comprising emitting said acoustic energy from an acoustic energy source placed at a location selected from the group consisting of a percutaneous location and a transrectal location.

13. A medical apparatus to determine a tumor characteristic related to a body region of a patient, comprising:
a source of acoustic energy configured to radiate said acoustic energy into a body region of a patient, to produce mechanical irritation in said body region;
a detection unit that detects a first value of a characteristic value of the patient that is correlated with the irritation, at a first point in time, and that detects a second value of the characteristic value at a second point in time following the first point in time and following the exposure with acoustic energy; and
an evaluation unit supplied with said first and second values, said evaluation unit being configured to determine a tumor characteristic from a change between said first value and said second value.

14. A medical apparatus as claimed in claim 13 comprising an imaging system that localizes said body region to align said source of acoustic energy with said body region.

15. A medical apparatus as claimed in claim 14 wherein said imaging apparatus is an ultrasound imaging apparatus.

16. A medical apparatus as claimed in claim 13 wherein said detection unit is an in vitro test unit.
